(19) United States
(12) Patent Application Publication
(10) Pub. No.: US 2019/0343939 A1
(43) Pub. Date: Nov. 14, 2019

(54) PERSONALIZED VACCINES

(71) Applicant: Dana-Farber Cancer Institute, Inc., Boston, MA (US)

(72) Inventor: Donald Kufe, Wellesley, MA (US)

(21) Appl. No.: 16/475,578

(22) PCT Filed: Jan. 11, 2018
(86) PCT No.: PCT/US18/13302

§ 371 Date: Jul. 2, 2019

Related U.S. Application Data
(60) Provisional application No. 62/515,892, filed on Jun. 6, 2017.
(60) Provisional application No. 62/444,945, filed on Jan. 11, 2017.

(51) Int. Cl. A61K 39/00; C12N 15/02; A61P 35/00; C12N 15/88; C12N 5/0784; C12N 5/09; C12N 5/077; C12N 5/16

(52) U.S. Cl. CPC A61K 39/0011; C12N 15/02; A61P 35/00; C12N 15/88; C12N 5/0639; A61K 2039/55; C12N 5/0656; C12N 5/16; A61K 2039/5154; A61K 2039/53; C12N 5/0693

(57) ABSTRACT

The present invention provides compositions and methods for treating cancer.

### CROSS-REFERENCE TO RELATED APPLICATIONS

This application claims the benefit of U.S. Provisional Application No. 62/444,945, filed on 11 Jan. 2017, and U.S. Provisional Application No. 62/515,892, filed on Jun. 6, 2017; the entire contents of each of said applications are incorporated herein in their entirety by this reference.

### BACKGROUND OF THE INVENTION

Tumor cells express unique antigens that are potentially recognized by the host T cell repertoire and serve as potential targets for tumor immunotherapy. However, tumor cells evade host immunity because antigens are presented in the absence of costimulation, and tumor cells express inhibitory cytokines that suppress native antigen presenting and effector cell populations. Thus, a promising area of investigation is the development of cancer vaccines to reverse tumor associated anergy and to stimulate effector cells to recognize and eliminate malignant cells.

### SUMMARY OF THE INVENTION

The present invention is based, at least in part, on providing personalized vaccine compositions and methods.

In one aspect, a cell population comprising a dendritic cell fused to vehicle comprising a cDNA expression library derived from one or more tumor cells, optionally wherein the tumor cell and dendritic cell are autologous, is provided.

Numerous embodiments are further provided that can be applied to any aspect of the present invention and/or combined with any other embodiment described herein. For example, in one embodiment, tumor cell is cultured in vitro. In another embodiment, the cells are cultured using a 3D cell culture. In still another embodiment, the tumor cells are a spheroid or organoid. In yet another embodiment, the vehicle is a MHC I/II null cell, optionally wherein the MHC I/II null cell is a fibroblast or a cancer cell line. In another embodiment, the vehicle is 1) a vesicle or 2) a polymeric nanoparticle (NP), optionally wherein the NP is a dual NP comprising a DNA expression vector-cationic peptide nanocomplex (NC) surrounded by a polymeric NP. In still another embodiment, the polymer NP is a diblock polymeric NP or a tetrablock polymeric NP. In yet another embodiment, the cDNA expression library is one or more defined tumor antigens and/or neoantigens, optionally wherein the tumor antigens and/or neoantigens are identified using RNA-seq.

In another aspect, method of treating a tumor in a patient comprising administering to the patient a composition comprising a cell population comprising a dendritic cell obtained from the patient fused to a vehicle comprising a cDNA expression library derived from a tumor cell, optionally wherein the tumor cell and dendritic cell are autologous, is provided.

As described above, numerous embodiments are further provided that can be applied to any aspect of the present invention and/or combined with any other embodiment described herein. For example, in one embodiment, the tumor cell is cultured in vitro, optionally wherein the culture is a 3D cell culture. In another embodiment, the tumor cells are a spheroid or organoid. In still another embodiment, the vehicle is 1) a vesicle or 2) a polymeric nanoparticle (NP), optionally wherein the NP is a dual NP comprising a DNA expression vector-cationic peptide nanocomplex (NC) surrounded by a polymeric NP. In yet another embodiment, the polymer NP is a diblock polymeric NP or a tetrablock polymeric NP. In another embodiment, the cDNA expression library is one or more defined tumor antigens and/or neoantigens, optionally wherein the tumor antigens and/or neoantigens are identified using RNA-seq. In still another embodiment, the tumor is a solid tumor or a hematologic malignancy, optionally wherein the solid tumor is a breast tumor or a renal tumor or wherein the hematologic malignancy is acute myeloid leukemia (AML) or multiple myeloma (MM). In yet another embodiment, the method further comprises administering to the patient an indoleamine-2,3-dioxygenase (IDO) inhibitor and/or a hypomethylating agent. In another embodiment, the method further comprises administering to the patient an immunomodulatory agent. In still another embodiment, the immunomodulatory agent is lenalidomide, pomalinomide, or apremilast. In yet another embodiment, the method further comprises administering to the patient a checkpoint inhibitor. In another embodiment, the checkpoint inhibitor is a PD1, PDL1, PDL2, TIM3, or LAG3 inhibitor. In still another embodiment, the checkpoint inhibitor is a PD1, PDL1, TIM3, or LAG3 antibody. In yet another embodiment, the method further comprises administering to the patient an agent that target regulatory T cells. In another embodiment, the method further comprises administering to the patient a TLR agonist, CPG ODN, polyIC, or tetanus toxoid. In still another embodiment, the hypermethylating agent is GO-203 or decitabine. In yet another embodiment, the IDO inhibitor is INB024360 or 1-MDT.

In still another aspect, a method of producing a fused cell population, comprising providing a population a population of dendritic cells (DC) or hyperactive dendritic cells and a vehicle comprising a cDNA expression library derived from a tumor cell and mixing the population of dendritic cells and the vehicle under conditions capable of mediating fusion of the dendritic cells and vehicle to produce a fused cell population, optionally wherein the tumor cell and dendritic cell are autologous, is provided.

As described above, numerous embodiments are further provided that can be applied to any aspect of the present invention and/or combined with any other embodiment described herein. For example, in one embodiment, the population of hyperactive dendritic cells is produced by a) contacting a population of dendritic cells with a composition comprising CpG DNA or LPS for a first period of time to produce a primed population of dendritic cells; and b) contacting the primed population of dendritic cells with a composition comprising oxidized phospholipids for a second period of time to produce a population of hyperactive dendritic cells. In another embodiment, the tumor cell is cultured in vitro. In still another embodiment, the cells are cultured using a 3D cell culture. In yet another embodiment, the tumor cells are a spheroid or organoid. In another embodiment, the vehicle is 1) a vesicle or 2) a polymeric nanoparticle (NP), optionally wherein the NP is a dual NP comprising a DNA expression vector-cationic peptide nanocomplex (NC) surrounded by a polymeric NP. In still another embodiment, the polymer NP is a diblock polymeric NP or a tetrablock polymeric NP. In yet another embodiment, the cDNA expression library is one or more defined tumor antigens and/or neoantigens, optionally wherein the tumor antigens and/or neoantigens are identified using RNA-seq. In another embodiment, the dendritic cells and the vehicles are at a ratio of 10:1 to 3:1. In still another embodiment, the conditions capable of mediating fusion include a fusion agent, optionally wherein the fusion agent is polyethylene glycol (PEG). In yet another embodiment, the method further comprises contacting the fused cell population with an indoleamine-2,3-dioxygenase (IDO) inhibitor.

In yet another aspect, a cell population produced by a method described herein, is provided. In one embodiment, the cell population is substantially free of endotoxin, microbial contamination and mycoplasma, optionally wherein the viability of the cell population is at least 80%.

In another aspect, a cell population comprising a dendritic cell fused to tumor cell, wherein the tumor cell is derived from 3D culturing a tumor cell obtained from a patient, optionally wherein the 3D culturing produces a tumor spheroid or organoid, is provided. In one embodiment, the cell population, wherein the dendritic cell and the tumor cell are autologous.

In still another aspect, a vaccine composition comprising a cell population described herein, is provided.

In yet another aspect, a method of treating a tumor in a patient comprising administering to the patient a composition comprising a cell population comprising a dendritic cell fused to tumor cell, wherein the tumor cell is derived from 3D culturing a tumor cell obtained from the patient, is provided.

As described above, numerous embodiments are further provided that can be applied to any aspect of the present invention and/or combined with any other embodiment described herein. For example, in one embodiment, the tumor is a solid tumor or a hematologic malignancy. In another embodiment, the solid tumor is a breast tumor, or a renal tumor. In still another embodiment, the hematologic malignancy is acute myeloid leukemia (AML) or multiple myeloma (MM). In yet another embodiment, the method further comprises administering to the patient an indoleamine-2,3-dioxygenase (IDO) inhibitor and/or a hypomethylating agent. In another embodiment, the method further comprises administering to the patient an immunomodulatory agent. In still another embodiment, the immunomodulatory agent is lenalidomide pomalinomide, or apremilast. In yet another embodiment, the method further comprises administering to the patient a checkpoint inhibitor. In another embodiment, the checkpoint inhibitor is a PD1, PDL1, PDL2, TIM3, or LAG3 inhibitor. In still another embodiment, the checkpoint inhibitor is a PD1, PDL1, TIM3, or LAG3 antibody. In yet another embodiment, the method further comprises administering to the patient an agent that target regulatory T cells. In another embodiment, the method further comprises administering to the patient a TLR agonist, CPG ODN, polyIC, or tetanus toxoid. In still another embodiment, the hypermethylating agent is GO-203 or decitabine. In yet another embodiment, the IDO inhibitor is INB024360 or 1-MDT.

In another aspect, a method of producing a fused cell population, comprising: providing a population a population of dendritic cells (DC) or hyperactive dendritic cells and a population of tumor spheroids or organoids and mixing the population of dendritic cells and the population spheroids or organoids under conditions capable of mediating fusion of the dendritic cells and spheroids or organoids to produce a fused cell population, optionally wherein the tumor spheroids or organoids and dendritic cells are autologous, is provided.

As described above, numerous embodiments are further provided that can be applied to any aspect of the present invention and/or combined with any other embodiment described herein. For example, in one embodiment, the population of hyperactive dendritic cells is produced by a) contacting a population of dendritic cells with a composition comprising CpG DNA or LPS for a first period of time to produce a primed population of dendritic cells; and b) contacting the primed population of dendritic cells with a composition comprising oxidized phospholipids for a second period of time to produce a population of hyperactive dendritic cells. In another embodiment, wherein the spheroids or organoids are derived from a patient's tumor. In still another embodiment, the dendritic cells and the spheroids or organoids are at a ratio of 10:1 to 3:1. In yet another embodiment, the conditions capable of mediating fusion include a fusion agent, such as polyethylene glycol (PEG). In another embodiment, the method further comprises contacting the fused cell population with an indoleamine-2,3-dioxygenase (IDO) inhibitor.

In still another aspect, a cell population produced by a method described herein, optionally wherein the cell population is substantially free of endotoxin, microbial contamination and mycoplasma and/or wherein the viability of the cell population is at least 80%, is provided.

In yet another aspect, a vaccine composition comprising a cell population described herein, is provided.

### BRIEF DESCRIPTION OF FIGURES

FIG. 1 shows a schematic diagram of personalized molecular fusion cell vaccine generation.

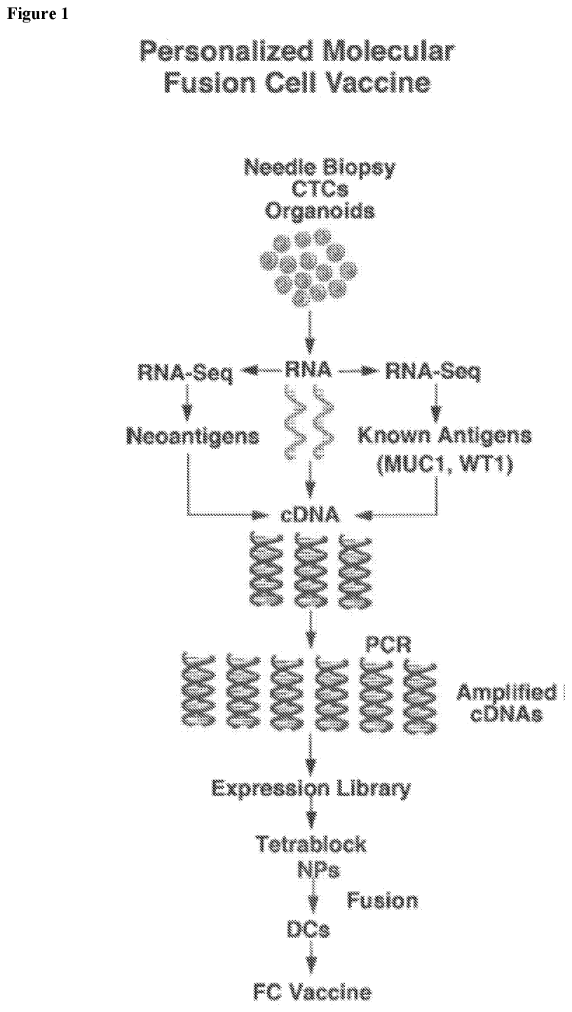

FIG. 2 shows an illustration of the production of cDNA containing polymeric nanoparticles.

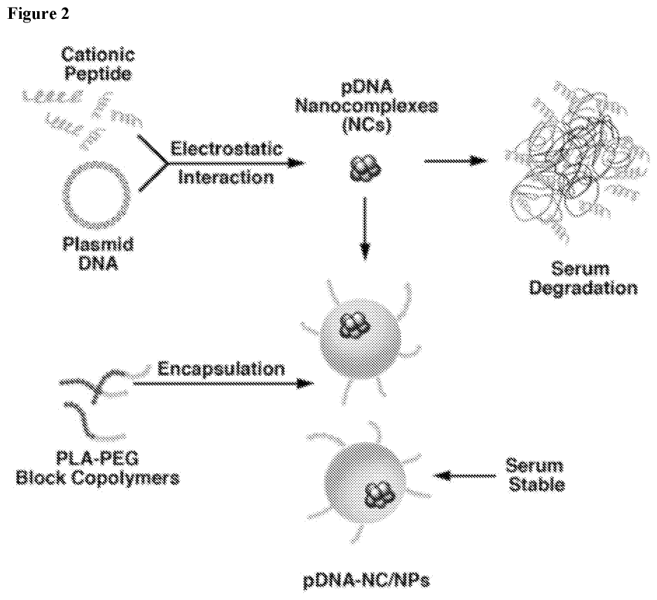

### DETAILED DESCRIPTION OF THE INVENTION

The present invention relates, in part, to methods for the treatment of tumors by administering to a subject a vaccine or immunogenic composition comprising dendritic cell fusions. More specifically, the present invention features immune system-stimulating compositions that contain cells formed by fusion between a dendritic cell (DCs) and vehicle expressing/containing tumor antigens. Fusions of tumor and dendritic cells have been effective in the treatment of patients with various cancers, such as multiple myeloma, acute myeloid leukemia (AML) and kidney cancer. However, a major limitation of this personalized vaccine strategy is the requirement of a large number of viable tumor cells for vaccine preparation. This is especially true for solid tumors where ample numbers of viable tumor cells are not readily available. The present invention provides a solution to this problem. An ideal fusion cell vaccine is one that can be generated from small amounts of tumor cells, even, for example, from a single cell such a circulating tumor cell (CTC).

Accordingly, the present invention is based upon generating a cDNA expression library from a single tumor stem cell or a pool of tumor cells. The cDNA expression library derived from the repertoire of tumor antigens is then introduced into a vehicle for fusion with a dendritic cell (DC). The vehicle is preferably a diblock or tetrablock polymeric nanoparticle (NP) that has been constructed to deliver cDNA expression constructs into the interior of the DC.

Specifically, a personalized molecular fusion cell vaccine is generated from a single or multiple tumor cells for example by (1) isolating mRNA: (2) reverse transcription of the mRNA and second strand synthesis (cDNA); (3) amplification of the cDNA; (4) generation of a cDNA expression library; (5) formation of a cDNA expression library-cationic nanocomplex (NC) surrounded by a diblock or tetrablock polymeric NP; (6) fusion of the cDNA-NC/NP with a DC that is autologous to the tumor cell.

Alternatively, the vehicle is a vesicle or a MHC I/II null cell. As used herein “cell fusion” is meant to include fusions between a dendritic cell and a cDNA nanoparticle, a cDNA vesicle, or a MHC I/II null cell expressing tumor antigens. For example, the cDNA library is introduced into (i) a vesicle and then fused with a DC that is autologous to the tumor cell, or (ii) a cell which is null for MHC I and II molecules; and fusion of the MHC I/II null cell with a dendritic cell that is autologous to the tumor cell.

The MHC I/II null cell can be any cell, such as a fibroblast or a cancer cell line, in which MHC genes have been silenced for example using gene editing techniques, such as the use of CRISPR/Cas9 gene editing techniques.

The present invention also contemplates tumor/dendritic cell fusions where the tumor cell(s) obtained from the subject are cultured in vitro to increase the number of tumor cells available for fusion.

### Tumor Cells

The tumor cells contemplated for use in connection with the present invention include, but are not limited to, tumor cells from breast cancer cells, ovarian cancer cells, pancreatic cancer cells, prostate gland cancer cells, renal cancer cells, lung cancer cells, urothelial cancer cells, colon cancer cells, rectal cancer cells, or hematological cancer cells. For example, hematological cancer cells include, but are not limited to, acute myeloid leukemia cells, acute lymphoid leukemia cells, multiple myeloma cells, and non-Hodgkin's lymphoma cells. Moreover, those skilled in the art would recognize that any tumor cell may be used in any of the methods of the present invention.

In some aspects, the tumor cells used in producing the cDNA expression library or fusions in accordance with the methods of the present invention include tumor cells obtained directly from a subject. Alternatively, tumor cells obtained from a subject may be cultured in vitro, prior to producing the cDNA library or fusion. Culturing the tumor cells is particularly useful when a sufficient number of tumor cells cannot be obtained from the subject sample. Any in vitro culturing technique may be utilized. Preferably, three dimensional (3D) culturing techniques are utilized to produce spheroids or organoid tumor cultures. Cells grown in 3D cultures systems to produce spheroids or organoids more closely resemble in vivo tissue in terms of cellular communication the development of extracellular matrices and tumor associated antigens.

3D culturing methods to produce tumor spheroids or organoids are well-known in the art. For example, the 3D culturing methods may utilize scaffold techniques or scaffold-free techniques.

Scaffold techniques include the use of solid scaffolds, hydrogels and other materials. Hydrogels are composed of interconnected pores with high water retention, which enables efficient transport of, e.g., nutrients and gases. Several different types of hydrogels from natural and synthetic materials are available for 3D cell culture, including, e.g., animal ECM extract hydrogels, protein hydrogels, peptide hydrogels, polymer hydrogels, and wood-based nanocellulose hydrogels.

Scaffold-free techniques employ another approach independent from the use of a scaffold. Scaffold-free methods include, for example, the use of low adhesion plates, hanging drop plates, micropatterned surfaces, rotating bioreactors, magnetic levitation, and/or magnetic 3D bioprinting.

In one embodiment, the patient has undergone therapy for the cancer. In another embodiment, the patient is in post-chemotherapy induced remission. In still another embodiment, the patient has had surgery to remove all or part of the tumor. For example, if the patient has multiple myeloma the patient may have an autologous stem cell transplant 30 to 100 days prior to the administration of the hyperactive cell fusions. If the patient has renal cell carcinoma, the patient may have a de-bulking nephrectomy prior to the administration of the cell fusions. if the patient has AML, then administration of the cell fusions follows induction of a complete remission with chemotherapy.

cDNA Libraries

As disclosed herein, vehicles of the present invention comprise elements of a cDNA expression library and the present invention is based, in part, upon generating a cDNA expression library from a single tumor stem cell or a pool of tumor cells. The preparation of cDNA libraries is well-known in the art. Gene expression in single cells and/or a plurality of single cells has previously been analyzed using a variety of methods (Brail et al. Mutat Res 406(2-4):45-54 (1999); Levsky et al. Science 297(5582):836-40 (2002); Bengtsson et al. Genome Res 15(10):1388-92 (2005); Esumi et al. Nat Genet 37(2):171-6 (2005)). For example, single cell gene expression in neural cells has been studied by microarray analysis (see Esumi et al. Neurosci Res 60(4):439-51 (2008)).

Generally, and as a non-limiting example, RNA is isolated from cells and mixed with deoxyribonuclease (DNase) to reduce amount of genomic DNA. The amount of RNA degradation may be assessed with, for example, gel and capillary electrophoresis and is used to assign an RNA integrity number to the sample. Quality and the total amount of starting RNA are taken into consideration during the subsequent library preparation, sequencing, and analysis steps.

Desirable RNA is then isolated, e.g. filtered for RNA with 3′ polyadenylated (poly(A)) tails to include only mRNA, depleted of ribosomal RNA (rRNA), and/or filtered for RNA that binds specific sequences. RNA with 3′ poly(A) tails typically represent mature, processed, coding sequences.

The RNA is reverse transcribed to cDNA followed by size selection to purify sequences of appropriate length. The nucleic acids (RNA and/or DNA) can be fragmented with enzymes, sonication, nebulizers, and the like, if desired. Fragmentation can also be followed by size selection, where either small sequences are removed or a tight range of sequence lengths may be selected. cDNA can be indexed with a hexamer or octamer barcode, so that they can be pooled.

The present invention provides methods and compositions for the analysis of gene expression in single tumor cells or in a plurality of tumor cells. In particular, the present invention provides methods for preparing a cDNA library from a plurality of single tumor cells. The methods are based on determining gene expression levels from a population of individual tumor cells, which can be used to identify natural variations in gene expression at a cell by cell level. The techniques can further be used to identify and characterize the cellular composition of a population of cells in the absence of suitable cell-surface markers. Said method also provides the advantage of generating a cDNA library representative of RNA content in a cell population by using single cells. Where known markers are available, these can be used to delineate cells of interest. Such cDNA libraries prepared by classical methods typically require total RNA isolated from a large population.

The present invention further contemplates a method of preparing a cDNA library from a plurality of single cells by releasing mRNA from each single cell to provide a plurality of individual samples, wherein the mRNA in each individual mRNA sample is from a single cell, synthesizing a first strand of cDNA from the mRNA in each individual mRNA sample and incorporating a tag into the cDNA to provide a plurality of tagged cDNA samples, wherein the cDNA in each tagged cDNA sample is complementary to mRNA from a single cell pooling the tagged cDNA samples and amplifying the pooled cDNA samples to generate a cDNA library comprising double-stranded cDNA. Thus, it is feasible to prepare samples for sequencing from several hundred single cells in a short time and with a minimal amount of work. Traditional methods for preparing a fragment library from RNA for sequencing include gel excision steps that are laborious. In the absence of special equipment, it is not convenient to prepare more than a handful of samples in parallel, thus technical variation is minimized.

In some aspects of the present invention, each cDNA sample obtained from a single cell is tagged, which allows expression analysis at the level of a single cell and of dynamic processes, such as the cell cycle, to be studied and distinct cell types in a complex tissue (e.g. tumors) to be analyzed. In some aspects of the present invention, the cDNA samples can be pooled prior to analysis. Pooling the samples simplifies handling of the samples from each single cell and reduces the time required to analyze gene expression in the single cells, allowing for high throughput analysis of gene expression. Pooling of the cDNA samples prior to amplification also provides the advantage that technical variation between samples is virtually eliminated. In addition, as the cDNA samples are pooled before amplification, less amplification is required to generate sufficient amounts of cDNA for subsequent analysis compared to amplifying and treating cDNA samples from each single cell separately. This reduces amplification bias, and means that any bias will be similar across all the cells used to provide pooled cDNA samples. RNA purification, storage and handling are also not required, which helps to eliminate problems caused by the unstable nature of RNA.

Before, concurrently with, or after cDNA expression library generation, various techniques well-known in the art can be applied to analyze gene expression profiles regarding the cDNA, such as microarray, parallel sequencing, nanopore sequencing, subtractive hybridization, and the like. In one embodiment, RNA-seq techniques can be used to analyze gene expression profiles of individual tumor cells. For example, RNA-seq can be used to analyze the fluctuating transcriptome of a cell or population of cells affording the ability to look at alternative gene spliced transcripts, post-transcriptional modifications, gene fusion, mutations/SNPs, temporal changes in gene expression, and/or differences in gene expression in different groups or treatment methodologies. Fragmentation, tagging, selection, and other useful techniques related to RNA-seq and other gene expression profiling methods are described above.

### Antigens

Although entire cDNA expression libraries from one or a few cells can be generated as described above, the term “cDNA expression library” includes the use of one or more particular cDNAs for expression that are less than the full complement of cDNAs cloned from a cell source. For example, cDNA expression libraries for use according to the present invention contemplated herein can be one or more tumor antigens of interest that are then introduced into a vehicle for fusion with a dendritic cell (DC). Accordingly, the instant invention contemplates populations of vehicle and/or cell fusions comprising, for example, known tumor antigens. Examples of common shared tumor antigens include proteins and/or peptides otherwise expressed only during embryonic development (e.g., cancer testis antigens such as NY-ESO-1 and SP17) (Batchu et al. (2005) Cancer Res. 65, 10041-10049; Dabadayev et al. (2005) Am. J. Hematol. 80, 6-11), peptides aberrantly or preferentially expressed by malignant cells (e.g., MUC1 in acute myelogenous leukemia and multiple myeloma (Koido et al. (2014) Anticancer Res. 34, 3917-3924) or BCMA, which is selectively expressed by B-lymphocytes and plasma cells (Carpenter et al. (2013) Clin. Cancer Res. 19, 2048-2060), or antigens truly unique to the tumor cell, such as the idiotype protein arising from the variable region of the immunoglobulin gene (Yi et al. (2010) Br. J. Haematol. 150, 554-564). Additionally and/or alternatively, one or more particular tumor neoantigens, such as those generated by somatic DNA alterations and mutations in cancer cells, are contemplated for use as a cDNA expression library according to the the present invention (Carreno et al. (2015) Science 348, 803-808). Cancer testis antigens, for example, are particularly useful for DC-tumor vaccine creation because of their limited expression on normal tissues and high expression by malignant hematologic cells. Moreover, their mRNA can be incorporated into autologous DCs via electroporation with relative ease. For example, Liggins et al. (2010) Cancer Immun. 10:8 have demonstrated that mRNA from at least eight cancer testis antigen genes—SP17, PRAME, CSAGE, PASD1, CAGE/DDX53, CTAGE1, HAGE/DDX43, and PLU-1/JARD1B—are expressed across numerous human B and T cell lymphoma cell lines. Similar observations have been made regarding the expression of cancer testis antigens of the MAGE and SSX families in bone marrow biopsy specimens of human patients with multiple myeloma (Dhodapkar et al. (2003) Cancer Immun. 3, 9; Jungbluth et al. (2005) Blood 106, 167-174; Taylor et al. (2005) J. Immunother. 28, 564-575). Several cancer testis antigen vaccines have been tested, or are currently, in clinical trials for hematologic malignancies, with varying degrees of immunologic and clinical success, and are considered herein as Table 1 adapted from Weinstock et al. (2017) Mol. Ther. Methods Clin. Dev. 5:66-75.

TABLE 1

| | Number | | |
|---|---|---|---|
| Disease | of patients | Tumor Antigen | Reference |
| Follicular | 35 | tumor idiotype | Timmerman et al. (2002) Blood 99, |
| lymphoma | | | 1517-1526 |
| Follicular | 18 | heat-shocked, | Di Nicola et al. (2009) Blood 113, 18-27 |
| lymphoma | | irradiated | |
| (relapsed) | | tumor cells | |
| CLL | 12 | leukemia cell | Hus et al. (2008) Leukemia 22, 1007-1017 |
| (chronic | | lysate | |
| lymphocytic | | | |
| leukemia) | | | |
| CLL | 15 | leukemia cell | Palma et al. (2012) Cancer Immunol. |
| | | lysate, apoptotic | Immunother. 61, 865-879 |
| | | tumor bodies | |
| CLL | 9 | leukemia cell | Hus et al. (2005) Leukemia 19, 1621-1627 |
| | | lysate, apoptotic | |
| | | tumor bodies | |
| ATLL | 3 | tax peptide | Suehiro et al. (2015) Br. J. Haematol. |
| | | | 169, 356-367 |
| Multiple | 12 | tumor idiotype | Reichardt et al. (1999) Blood 93, 2411-2419 |
| Myeloma | | | |
| Multiple | 26 | tumor idiotype | Liso et al. (2000). Biol. Blood Marrow |
| Myeloma | | | Transplant. 6, 621-627 |
| Multiple | 27 | tumor idiotype | Lacy et al. (2009) Am. J. Hematol. 84, |
| Myeloma | | | 799-802. |
| Multiple | 12 | mRNA from | Hobo et al. (2013) Cancer Immunol. |
| Myeloma | | MAGE3, | Immunother. 62, |
| | | Survivin, | 1381-1392 |
| | | and BCMA | |
| Multiple | 18 | Whole tumor | Rosenblatt (2011) Blood 117, 393-402 |
| Myeloma | | cell | |
| Multiple | 36 | Whole tumor | Rosenblatt (2013) Clin. Cancer Res. 19, |
| Myeloma | | cell | 3640-3648 |
| Multiple | NA | Whole cell | Clinical Trials.gov. (2016). Dendritic |
| Myeloma | | tumor fusion | cell/myeloma fusion vaccine for |
| | | | multiple |
| | | | myeloma (BMT CTN 1401) |
| Multiple | NA | survivin | Clinical Trials.gov. (2016). Survivin |
| Myeloma | | | vaccine: multiple myeloma autologous |
| | | | hematopoietic |
| | | | cell transplant (HCT) |
| Multiple | NA | mRNA for CT7, | Clinical Trials.gov. (2016). CT7, |
| Myeloma | | MAGE-A3, and | MAGE-A3, and WT1 mRNA- |
| | | WT1 | electroporated autologous |
| | | | Langerhans-type dendritic cells as |
| | | | consolidation for multiple myeloma |
| AML (acute | 10 | mRNA from | Van Tendeloo (2010) Proc. Natl. Acad. |
| myelogenous | | WT1 | Sci. USA 107, 13824-13829 |
| leukemia) | | | |
| AML | 17 | whole tumor cell | Rosenblatt (2016) Sci. Transl. Med. 8, |
| | | | 368ra171 |
| AML | NA | mRNA for WT1 | Clinical Trials.gov. (2016) Efficacy |
| | | | study of dendritic cell vaccination in |
| | | | patients with acute myeloid leukemia in |
| | | | remission (WIDEA) |
| AML | NA | two leukemia | Clinical Trials.gov. (2016) DC |
| | | antigens and one | vaccination for post-remission therapy |
| | | CMV antigen | in AML |
| AML | NA | mRNA for WT1 | Clinical Trials.gov. (2016). DC |
| | | and PRAME | vaccination for post-remission therapy |
| | | | in AML |

The term “cDNA expression library” encompasses 1) a cDNA expression library generated from a single cell, such as cancer cell, 2) a cDNA expression library generated from a several or many cells, such as multiple cancer cells, 3) one ore more particular cDNAs, such as cDNAs expressing particular tumor antigen and/or neoantigens, and 4) combinations thereof, inclusive, such as a cDNA expression library generated from a single cell in combination with spiked in cDNAs for particular tumor antigen(s) and/or neoantigen(s) of interest.

### Nanoparticle Vehicles

Nanoparticles contemplated for use in connection with the present invention include, but are not limited to, particles in the range between 10 nm to 1000 nm in diameter, wherein diameter refers to the diameter of a perfect sphere having the same volume as the particle. The term “nanoparticle” is used interchangeably as “nanoparticle(s).” In some embodiments, the diameter of the particle is in the range of about 1-1000 nm, 10-500 nm, or 30-120 nm. In some embodiments, a population of particles can be present. As used herein, the “diameter” of the nanoparticles is an average of a distribution in a particular population.

The present invention provides a non-toxic, safe, biodegradable polymeric nanoparticle made up of copolymer and a process for preparing the same. The biodegradable polymeric nanoparticles of the instant invention can be formed of a block copolymer. Block copolymers comprise two or more homopolymer subunits linked by covalent bonds wherein the union of the homopolymer subunits may require an intermediate non-repeating subunit, known as a junction block. Block copolymers with two, three, or four distinct blocks are called diblock, triblock, and tetrablock copolymers, respectively. In some embodiments, the block copolymers consist essentially of poly (lactic acid) (PLA) chemically modified with a hydrophilic hydrophobic block copolymer, wherein said hydrophilic-hydrophobic block copolymer is selected from poly(methyl methacrylate)-poly(methylacrylic acid) (PMMA-PMAA), poly(styrene)-poly(acrylic acid) (PS-PAA), poly(acrylicacid)-poly(vinylpyridine) (PAA-PVP), poly(acrylic acid)-poly(N,N-dimethylaminoethyl methacrylate) (PAA-PD MAEMA), poly(ethylene glycol)-poly(butylene glycol) (PEG-PBG), and poly(ethylene glycol)-poly(propylene glycol)-poly(ethylene glycol) (PEG-PPG-PEG).

The present invention provides a process for preparing the biodegradable polymeric nanoparticle of the present invention. The resulting nanoparticle is not only non-toxic, safe, and biodegradable, but is stable in vivo, has high storage stability, and can be safely used in a nanocarrier system or drug delivery system in the field of medicine. The present invention also provides a process for efficient drug loading on a biodegradable polymeric nanoparticle to form an effective and targeted drug delivery nanocarrier system which prevents premature degradation of active agents and/or nucleic acids and has a strong potential for use in cancer therapy.

Preferably, the nanoparticle is a dual nanoparticle (DNP) system for carrying cDNAs and/or antigens into cells, such as dendritic cells. The DNP system may comprise a DNA expression vector-cationic peptide nanocomplex (NC) surrounded by a diblock polymeric NP. In other embodiments the NC is surrounded by a tetrablock polymeric NP.

In preferred embodiments, nanocomplexes (NCs) are generated by incubation of a positively charged co-packaging peptide with a negatively charged expression vector. In some such embodiments, the co-packaging peptide is designed for maintaining the balance of DNA condensation and intracellular release and nuclear localization (Mann et al. (2014) Mol Pharm 11(3):683-96). To circumvent the instability of the expression vector in plasma, a dual nanocomplex in a nanoparticle system can be generated by encapsulation of NC (comprising peptide) in polymeric NPs. Intracellular delivery of the expression vector in NPs results in sustained release of the expression vector, import into the nucleus, and expression of the cDNA. Intratumoral expression of potentially therapeutic sequences is useful for inducing anti-tumor activity and potentially reprogramming the tumor immune microenvironment.

In certain embodiments, the co-packaging peptide comprises a histidine and cysteine modified arginine (HCR) peptide. In a preferred embodiment, the co-packaging peptide component of the NC is a histidine and cysteine modified arginine (HCR) peptide. For delivery in vivo, the NC is surrounded by a polymer diblock NP to circumvent the challenge of DNA (or antigen)-nanoparticle serum interactions (Zagorovsky et al. (2016) Proc Natl Acad Sci USA 113(48):13600-5). In some such embodiments, the DNP system may be broadly applicable for expression of cancer-specific antigens and/or intratumoral cytokines that promote immune recognition and destruction. By way of example and without seeking limitation, HCR-pDNA nanocomplexes (NC) exploit the electrostatic interaction of amino nitrogen (NH3+) of peptide per phosphate (PO4−) group of DNA at a charge ratio [Z(±)] of 10.0. The pDNA stock can be diluted to a concentration of 20-40 ng/μl and added drop wise to an equal volume of HCR peptide dilutions while vortexing.

In some embodiments, NPs are prepared using a double emulsion evaporation method (Shi et al. (2013) J Biol Eng 7(1):25), wherein polymer solution and surfactant were prepared as: a) PEG-PLA block co-polymer dissolved in acetonitrile at a concentration of 6 mg/ml and b) Ploxomer F-127 dissolved at 3 mg/ml in de-ionized water with continuous stirring. In some such embodiments, pDNA-NCs are gently mixed with the PLA-PEG solution and then added slowly into the aqueous surfactant solution. In further embodiments, a solution can be continuously stirred for approximately 12 h to form a stable NP solution. In some such embodiments, the block copolymer is a diblock copolymer, such as PLA-PEG. In other embodiments, the block copolymer is a tetrablock copolymer, such as PLA-PEG-PPG-PEG.

Representative diblock copolymers, tetrablock copolymers, HCR-pDNA nanocomplexes, and other nanoparticles useful for use according to the present invention are described in U.S. Pat. Publ. 2015/0353676 and Shukla et al. (2017) Nanomed. Nanotechnol. Biol. Med. 13:1833-1839.

### Dendritic Cells

DCs can be obtained from bone marrow cultures, peripheral blood, spleen, or any other appropriate tissue of a mammal using protocols known in the art. Bone marrow contains DC progenitors, which, upon treatment with cytokines, such as granulocyte-macrophage colony-stimulating factor (“GM-CSF”) and interleukin 4 (“IL-4”), proliferate and differentiate into DCs. Tumor necrosis cell factor (TNF) is optionally used alone or in conjunction with GM-CSF and/or IL-4 to promote maturation of DCs. DCs obtained from bone marrow are relatively immature (as compared to, for instance, spleen DCs). GMCSF/IL-4 stimulated DC express MHC class I and class II molecules, B7-1, B7-2, ICAM, CD40 and variable levels of CD83. These immature DCs are more amenable to fusion (or antigen uptake) than the more mature DCs found in spleen, whereas more mature DCs are relatively more effective antigen presenting cells. Peripheral blood also contains relatively immature DCs or DC progenitors, which can propagate and differentiate in the presence of appropriate cytokines such as GM-CSF and-which can also be used in fusions.

Preferably, the DCs are obtained from peripheral blood. For example, the DCs are obtained from the patients' peripheral blood after it has been documented that the patient is in complete remission.

The DC can be made hyperactive prior to fusion or after fusion. DCs can be made hyperactive by any method know in the art. For example, DCs are made hyperactive by contacting the DC or DC fusion with a priming agent followed by an activating agent. Exemplary priming agents include CpG DNA or LPS. Activating agents include for example oxidized phospholipids.

In some embodiments, DCs have sufficient viability prior to fusion, such as at least 70%, at least 75%, at least 80%, or greater.

Prior to fusion, the population of the DCs are generally free of components used during the production, e.g., cell culture components and substantially free of mycoplasm, endotoxin, and microbial contamination. Preferably, the population of DCs has less than 10, 5, 3, 2, or 1 CFU/swab. Most preferably, the population of DCs has 0 CFU/swab.

Prior to fusion, the population of vehicles expressing tumor antigens is generally free of components used during the isolation and substantially free of mycoplasm endotoxin, and microbial contamination. Preferably, the cell population has less than 10, 5, 3, 2, or 1 CFU/swab. Most preferably the population of cells has 0 CFU/swab. The endotoxin level in the population of cells is less than 20 EU/mL, less than 10 EU/mL or less than 5 EU/mL.

Prior to fusion, the population of tumor organoids or spheroids are generally free of components used during the isolation and substantially free of mycoplasm, endotoxin, and microbial contamination. Preferably, the cell population has less than 10, 5, 3, 2, or 1 CFU/swab. Most preferably, the population of cells has 0 CFU/swab. The endotoxin level in the population of cells is generally less than 20 EU/mL, less than 10 EU/mL, or less than 5 EU/mL.

The fusion product can be used directly after the fusion process (e.g., in antigen discovery screening methods or in therapeutic methods) or after a short culture period.

The hyperactive cell fusions can be irradiated prior to clinical use. Irradiation induces expression of cytokines, which promotes immune effector cell activity. Irradiation also prevents the cells from replicating, thereby reducing or eliminating any risk of oncogenesis.

In the event that the fused DCs lose certain DC characteristics, such as expression of the APC-specific T-cell stimulating molecules, primary fused cells can be re-fused with dendritic cells to restore the DC phenotype. The re-fused cells (i.e., secondary fused cells) are found to be highly potent APCs. The fused cells can be re-fused with the dendritic or non-dendritic parental cells as many times as desired.

Cell fusions that express MHC class II molecules, B7, or other desired T-cell stimulating molecules can also be selected by panning or fluorescence-activated cell sorting with antibodies against these molecules.

Fusion can be carried out with well-known methods, such as those using polyethylene glycol (“PEG”) or electrofusion. Alternatively, the cDNA-NC/NPs or cDNA vesicles or tumor organoids or spheroids can fuse with DCs in the absence of PEG or electrofusion. DCs are autologous or allogeneic (see. e.g., U.S. Pat. No. 6,653,848, which is herein incorporated by reference in its entirety).

The ratio of DCs to MHC I/II null cells expressing tumor antigens in fusion can vary from 1:100 to 1000:1, with a ratio higher than 1:1 being preferred. Preferably, the ratio is 1:1, 5:1, or 10:1. Most preferably, the ratio of DCs to cells is 10:1 or 3:1.

Alternatively, the ratio of DCs to cDNA-NC/NPs encoding, expressing, or presenting tumor antigens can vary from about 2×10⁵ to about 1×10⁶ DCs to about 0.2 to 1.24 μg cDNA-NC/NPs. In some such embodiments the amount of DCs is 2×10⁵ and the amount of cDNA-NC/NPs is 0.2 μg (Kranz et al. (2016) Nature, 534:396). In yet a further alternative, the ratio of DCs to cDNA-vesicles encoding, expressing, or presenting tumor antigens can vary from about 2×10⁵ to about 1×10⁶ DCs to about 0.2 to 20 μg cDNA-vesicles. In some such embodiments the amount of DCs is 2×10⁵ and the amount of cDNA-vesicles is 20 μg (Ding G et al. (2015) Oncotarget 6:29877).

After fusion, unfused DCs usually die off in a few days in culture, and the fused cells can be separated from the unfused, parental, non-dendritic cells by the following two methods, both of which yield fused cells of approximately 50% or higher purity, i.e., the fused cell preparations contain less than 50%, and often less than 30%, unfused cells.

Isolation of Fused Cells

Specifically, one method of separating unfused cells/vehicles from fused cells is based on the different adherence properties between the fused cells and the vehicles or MHC I/II null cells expressing tumor antigens. It has been found that the fused cells are generally lightly adherent to tissue culture containers. Thus, if the cells expressing tumor antigens are much more adherent, the post-fusion cell mixtures can be cultured in an appropriate medium for a short period of time (e.g., 5-1 0 days).

Subsequently, cell fusions can be gently dislodged and aspirated off while the MHC I/II null cells expressing tumor antigens or are firmly attached to the tissue culture containers. Conversely, if the cells expressing tumor antigens are in suspension, after the culture period, they can be gently aspirated off while leaving the DC fusions loosely attached to the containers. Alternatively, the hybrids are used directly without an in vitro cell culturing step.

The cell fusions obtained by the above-described methods typically retain the phenotypic characteristics of DCs. For instance, these fusions express T-cell stimulating molecules such as MEW class II protein, B7-L B7-2, and adhesion molecules characteristic of APCs such as ICAM-I. The fusions also continue to express cell-surface antigens of the tumor cells such as MUCI, and are therefore useful for inducing immunity against the cell surface antigens.

In the event that the fusions lose certain DC characteristics such as expression of the APC-specific T-cell stimulating molecules, they (i.e., primary fusions) can be re-fused with dendritic cells to restore the DC phenotype. The re-fused cells (i.e., secondary fusions) are found to be highly potent APCs, and in some cases, have even less tumorigenicity than primary fusions. The fusions can be re-fused with the dendritic cell as many times as desired. The DCs can be made hyperactive prior to or after re-fusion.

The cell fusions can be frozen before administration. The fusions can be frozen in a solution containing 10% DMSO in 90% heat inactivated autologous plasma.

### Immune Therapy

The cell fusions of the present invention can be used to stimulate the immune system of a mammal for treatment or prophylaxis of cancer. For instance, to treat cancer in a human, a composition containing cell fusions formed by their own DCs can be administered to them, e.g., at a site near the lymphoid tissue. Preferably, the vaccine is administered to four different sites near lymphoid tissue. The composition may be given multiple times (e.g., two to five, preferably three) at appropriate intervals, preferably, four weeks and dosage (e.g., approximately 10⁵-10⁸, e.g., about 0.5×10⁶ to 1×10⁶, cell fusions per administration). Preferably, each dosage contains approximately 1×10⁶ to 1×10⁷ cell fusion. More preferably each dosage contains approximately 5×10⁶ cell fusions. In some embodiments, in addition to the cell fusions, the patient further receives GM-CSF. The GM-CSF can be administered on the day the cell fusions are administered daily for three subsequent days. The GM-CSF can be administered subcutaneously at a dose of 100 μg. The GM-CSF can be administered at the site where the cell fusions are administered.

In some embodiments, the patient further receives an immunomodulatory drug, such as thalidomide, lenalidomide, pomalidomide, or apremilast. The immunomodulatory drug can be administered at a therapeutic dose. For example, the patient can receive 5 mg, 10 mg, 15 mg, 20 mg, 25 mg or more per day. In other aspects, the immunomodulatory drug can be administered at a sub-therapeutic dose. The term “sub-therapeutic dose” refers to a level that is below the level typically necessary to treat disease.

In some embodiments, the patient can further receive a checkpoint inhibitor. For example, the checkpoint inhibitor can be administered contemporaneously with the fused cell, prior to administration of the fused cells, or after administration of the fused cells. In some embodiments, the checkpoint inhibitor is administered 1 week prior to the fused cells. Preferably, the checkpoint inhibitor is administered 1 week after the fused cells. In some embodiments, the checkpoint inhibitor can be administered at 1, 2, 3, 4, 5, 6 week intervals.

By checkpoint inhibitor it is meant that the compound inhibits a protein in the checkpoint signaling pathway. Proteins in the checkpoint signaling pathway include for example, PD-1, PD-L1, PD-L2, TIM3, LAG3, and CTLA-4. Immune checkpoint inhibitors are known in the art. For example, the checkpoint inhibitor can be a small molecule. A “small molecule” as used herein, is meant to refer to a composition that has a molecular weight in the range of less than about 5 kD to 50 daltons, for example less than about 4 kD, less than about 3.5 kD, less than about 3 kD, less than about 2.5 kD, less than about 2 kD, less than about 1.5 kD, less than about 1 kD, less than 750 daltons, less than 500 daltons, less than about 450 daltons, less than about 400 daltons, less than about 350 daltons, less than 300 daltons, less than 250 daltons, less than about 200 daltons, less than about 150 daltons, less than about 100 daltons. Small molecules can be, e.g., nucleic acids, peptides, polypeptides, peptidomimetics, carbohydrates, lipids or other organic or inorganic molecules.

Alternatively, the checkpoint inhibitor can be an antibody or fragment thereof. For example, the antibody or fragment thereof specifically binds to and inhibits an immune checkpoint protein, such such as PD-L PD-L1 PD-L2, TIM3, LAG3, or CTLA-4. Preferably, the checkpoint inhibitor is an antibody specific for PD-L PD-L1 PD-L2, TIM3, LAG3, or CTLA-4.

In some embodiments, the patient can be administered a hypomethylating agent (HMA). A HMA includes for example, GO-203 or decitabine.

In some embodiments, the patient can be administered an indoleamine 2, 3-dioxygenase (IDO) inhibitor. IDO inhibitors are known in the art and include for example INCB024360 (indoximod) or 1-MDT (NLG8189).

To monitor the effect of vaccination, cytotoxic T lymphocytes obtained from the treated individual can be tested for their potency against cancer cells in cytotoxic assays. Multiple boosts may be needed to enhance the potency of the cytotoxic T lymphocytes.

Compositions containing the appropriate cell fusions can be administered to an individual (e.g., a human) in a regimen determined as appropriate by a person skilled in the art. For example, the composition can be given multiple times (e.g., three to five times, preferably three) at an appropriate interval (e.g., every four weeks) and dosage (e.g., approximately 10⁵-10⁵, preferably about 1×10⁶ to 1×10⁷, more preferably 5×10⁶ cell fusions per administration).

The composition of cell fusions prior to administration to the patient generally have sufficient viability. For example, the viability of the fused cells at the time of administration can be at least 50%, at least 60%, at least 70%, at least 80%, or greater.

Prior to administration, the population of cell fusions are generally free of components used during the production, e.g., cell culture components, and substantially free of mycoplasm, endotoxin, and microbial contamination. Preferably, the population of cell fusions has less than 10, 5, 3, 2, or 1 CPU/swab. Most preferably, the population of cell fusions has 0 CFU/swab. For example, the results of the sterility testing can be “negative” or “no growth.” The endotoxin level in the population of cell fusions can be less than 20 EU/mL, less than 10 EU/mL, or less than 5 EU/mL. The results of the mycoplasm testing can be “negative”.

Prior to administration, the cell fusions generally express at least 40%, at least 50%, at least 60% CD86, or more, as determined by immunological staining. Preferably the fused cells express at least 50% CD86.

More specifically, final cell product generally conforms with requirements imposed by the Federal Drug Administration (FDA). The FDA requires that all final cell products minimize “extraneous” proteins known to be capable of producing allergenic effects in human subjects as well as minimize contamination risks. Moreover, the FDA expects a minimum cell viability of 70%, and any process should consistently exceed this minimum requirement.

### Definitions

The practice of the present invention employs, unless otherwise indicated, conventional techniques of molecular biology, microbiology, cell biology and recombinant DNA, which are within the skill of the art (See, e.g., Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel et al. eds., (1987)); the series METHODS TN ENZYMOLOGY (Academic Press, Inc.): PCR 2: A PRACTICAL APPROACH (Mi. MacPherson, B D. Hames and G. R Taylor eds. (1995)) and ANIMAL CELL CULTURE (Rd. Freshney, ed. (1987)).

As used herein, certain terms have the following defined meanings. As used in the specification and claims, the singular form “a”, “an” and “the” include plural references unless the context clearly dictates otherwise. For example, the term “a cell” includes a plurality of cells, including mixtures thereof.

The term “immune effector cells” refers to cells that specifically recognize an antigen present, for example on a neoplastic or tumor cell. For the purposes of the present invention, immune effector cells include, but are not limited to, B cells; monocytes; macrophages; NK cells; and T cells such as cytotoxic T lymphocytes (CTLs), for example CTL lines, CTL clones, and CTLs from tumor, inflammatory sites or other infiltrates. The term “T-lymphocytes” denotes lymphocytes that are phenotypically CD3+, typically detected using an anti-CD3 monoclonal antibody in combination with a suitable labeling technique. The T-lymphocytes of the present invention are also generally positive for CD4, CD8, or both. The term “naive” immune effector cells refers to immune effector cells that have not encountered antigen and is intended to by synonymous with unprimed and virgin. The term “educated” refers to immune effector cells that have interacted with an antigen such that they differentiate into an antigen-specific cell.

The terms “antigen presenting cells” or “APCs” include both intact whole cells as well as other molecules which are capable of inducing the presentation of one or more antigens, preferably with class I MHC molecules. Examples of suitable APCs are discussed in detail below and include, but are not limited to, whole cells, such as macrophages, dendritic cells, B cells; purified MHC class I molecules complexed to J32-microglobulin; and foster antigen presenting cells.

Dendritic cells (DCs) are potent APCs. DCs are minor constituents of various immune organs, such as spleen, thymus, lymph node, epidermis, and peripheral blood. For instance, DCs represent merely about 1% of crude spleen (see Steinman et al. (1979) J. Exp. Med 149: 1) or epidermal cell suspensions (see Schuler et al. (1985) J. Exp. Med 161:526; Romani et al. J Invest. Dermatol (1989) 93: 600) and 0.1-1% of mononuclear cells in peripheral blood (see Freudenthal et al. Proc. Natl Acad Sci USA (1990) 87: 7698). Methods for isolating DCs from peripheral blood or bone marrow progenitors are known in the art (see Inaba et al. (1992) J. Exp. Med 175:1157; Inaba et al. (1992) J. Exp, Med 176: 1693-1702; Romani et al. (1994) J. Exp. Med. 180: 83-93; Sallusto et al. (1994) J. Exp. Med 179: 11 09-1118). Preferred methods for isolation and culturing of DCs are described in Bender et al. (1996) J. Immun. Meth. 196:121-135 and Romani et al. (1996) J. Immun. Meth 196:137-151.

Dendritic cells (DCs) represent a complex network of antigen presenting cells that are primarily responsible for initiation of primary immunity and the modulation of immune response (see Avigan, Blood Rev. 13:51-64 (1999); Banchereau et al. Nature 392:245-52 (1998)). Partially mature DCs are located at sites of antigen capture and excel at the internalization and processing of exogenous antigens, but are poor stimulators of T cell responses. Presentation of antigen by immature DCs may induce T cell tolerance (see Dhodapkar et al. J Exp Med. 193:233-38 (2001)). Upon activation, DCs undergo maturation characterized by the increased expression of costimulatory molecules and CCR7, the chemokine receptor which promotes migration to sites of T cell traffic in the draining lymph nodes. Tumor or cancer cells inhibit DC development through the secretion of IL-10, TGF-β), and VEGF resulting in the accumulation of immature DCs in the tumor bed that potentially suppress anti-tumor responses (see Allavena et al, Eur. J. Immunol. 28:359-69 (1998); Gabrilovich et al. Clin Cancer Res. 3:483-90 (1997); Gabrilovich et al. Blood 92:4150-66 (1998); Gabrilovich, Nat Rev Immunol 4:941-52 (2004)). Conversely, activated DCs can be generated by cytokine-mediated differentiation of DC progenitors ex vivo. DC maturation and function can be further enhanced by exposure to the toll-like receptor 9 agonist, CPG ODN. Moreover, DCs can be manipulated to present tumor antigens potently stimulate anti-tumor immunity (see Asavaroenhchai et al. Proc Natl Acad Sci USA 99:931-36 (2002); Ashley et al. J Exp Med 186:1177-82 (1997)).

The term “foster antigen presenting cells” refers to any modified or naturally occurring cells (wild-type or mutant) with antigen presenting capability that are utilized in lieu of antigen presenting cells (“APC”) that normally contact the immune effector cells with which they are to react. In other words, they are any functional APCs that T cells would not normally encounter in vivo.

It has been shown that DCs provide all the signals required for T cell activation and proliferation. These signals can be categorized into two types. The first type, which gives specificity to the immune response, is mediated through interaction between the T-cell receptor/CD3 (“TCR/CD3”) complex and an antigenic peptide presented by a major histocompatibility complex (“MHC”) class I or II protein on the surface of APCs. This interaction is necessary, but not sufficient, for T cell activation to occur. In fact, without the second type of signals, the first type of signals can result in T cell anergy. The second type of signals, called costimulatory signals, are neither antigen-specific nor MHC restricted, and can lead to a full proliferation response of T cells and induction of T cell effector fluctuations in the presence of the first type of signals.

Thus, the term “cytokine” refers to any of the numerous factors that exert a variety of effects on cells, for example, inducing growth or proliferation. Non-limiting examples of cytokines include, IL-2, stem cell factor (SCF), IL-3, IL-6, IL-7, IL-12, IL-15, G-CSF, GM-CSF, IL-1α, IL-1β, MIP-1α, LIF, c-kit ligand, TPO, and flt3 ligand. Cytokines are commercially available from several vendors such as, for example, Genzyme Corp. (Framingham, Mass.), Genentech (South San Francisco, Calif.), Amgen (Thousand Oaks, Calif.) and Immunex (Seattle, Wash.). It is intended, although not always explicitly stated, that molecules having similar biological activity as wild-type or purified cytokines (e.g., recombinantly produced cytokines) are intended to be used within the spirit and scope of the present invention and therefore are substitutes for wild-type or purified cytokines.

“Costimulatory molecules” are involved in the interaction between receptor-ligand pairs expressed on the surface of antigen presenting cells and T cells. One exemplary receptor-ligand pair is the B7 co-stimulatory molecules on the surface of DCs and its counter-receptor CD28 or CTLA-4 on T cells (see Freeman et al. (1993) Science 262:909-911; Young et al. (1992) J. Clin. Invest 90: 229; Nabavi et al. Nature 360:266)). Other important costimulatory molecules include, for example, CD40, CD54, CD80, and CD86. These are commercially available from vendors identified above.

A “hybrid” cell refers to a cell having both antigen presenting capability and also expresses one or more specific antigens. In one embodiment, these hybrid cells are formed by fusing, in vitro, APCs with cells that are known to express the one or more antigens of interest. As used herein, the term “hybrid” cell and “fusion” cell are used interchangeably.

A “control” cell refers to a cell that does not express the same antigens as the population of antigen-expressing cells.

The term “culturing” refers to the in vitro propagation of cells or organisms on or in media of various kinds, it is understood that the descendants of a cell grown in culture may not be completely identical (i.e., morphologically, genetically, or phenotypically) to the parent cell. By “expanded” is meant any proliferation or division of cells.

An “effective amount” is an amount sufficient to effect beneficial or desired results. An effective amount can be administered in one or more administrations, applications or dosages. For purposes of this invention, an effective amount of hybrid cells is that amount which promotes expansion of the antigenic-specific immune effector cells, e.g, T cells.

An “isolated” population of cells is “substantially free” of cells and materials with which it is associated in nature. By “substantially free” or “substantially pure” is meant at least 50% of the population are the desired cell type, preferably at least 70%, more preferably at least 80%, and even more preferably at least 90%. An “enriched” population of cells is at least 5% fused cells. Preferably, the enriched population contains at least 10%, more preferably at least 20%, and most preferably at least 25% fused cells.

The term “autogeneic” or “autologous,” as used herein, indicates the origin of a cell. Thus, a cell being administered to an individual (the “recipient”) is autogeneic if the cell was derived from that individual (the “donor”) or a genetically identical individual (i.e., an identical twin of the individual). An autogeneic cell can also be a progeny of an autogeneic cell. The term also indicates that cells of different cell types are derived from the same donor or genetically identical donors. Thus, an effector cell and an antigen presenting cell are said to be autogeneic if they were derived from the same donor or from an individual genetically identical to the donor, or if they are progeny of cells derived from the same donor or from an individual genetically identical to the donor.

Similarly, the term “allogeneic”, as used herein, indicates the origin of a cell. Thus, a cell being administered to an individual (the “recipient”) is allogeneic if the cell was derived from an individual not genetically identical to the recipient. In particular, the term relates to non-identity in expressed MHC molecules. An allogeneic cell can also be a progeny of an allogeneic cell. The term also indicates that cells of different cell types are derived from genetically non-identical donors, or if they are progeny of cells derived from genetically non-identical donors. For example, an APC is said to be allogeneic to an effector cell if they are derived from genetically non-identical donors.

A “subject” can be a vertebrate, preferably a mammal, and more preferably, a human. Mammals include, but are not limited to, murines, simians, humans, farm animals, sport animals, and pets.

As used herein, “genetic modification” refers to any addition, deletion or disruption to a cell's endogenous nucleotides.

A “viral vector” is defined as a recombinantly produced virus or viral particle that comprises a poly nucleotide to be delivered into a host cell, either in vivo, ex vivo or in vitro. Examples of viral vectors include retroviral vectors, adenovirus vectors, adeno-associated virus vectors and the like. In aspects where gene transfer is mediated by a retroviral vector, a vector construct refers to the polynucleotide comprising the retroviral genome or part thereof and a therapeutic gene.

As used herein, the terms “retroviral mediated gene transfer” or “retroviral transduction” carries the same meaning and refers to the process by which a gene or a nucleic acid sequence is stably transferred into the host cell by virtue of the virus entering the cell and integrating its genome into the host cell genome. The virus can enter the host cell via its normal mechanism of infection or be modified such that it binds to a different host cell surface receptor or ligand to enter the cell.

Retroviruses carry their genetic information in the form of RNA. However, once the virus infects a cell, the RNA is reverse-transcribed into the DNA form that integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a therapeutic gene. Adenoviruses (Ads) are a relatively well characterized, homogenous group of viruses, including over 50 serotypes. (See, e.g., WO 95/27071). Ads are easy to grow and do not integrate into the host cell genome. Recombinant Ad-derived vectors, particularly those that reduce the potential for recombination and generation of wild-type virus, have also been constructed (see WO 95/00655 and WO 95/11984). Wild-type AAV has high infectivity and specificity integrating into the host cells genome (see Hermonat and Muzyczka (1984) PNAS USA 81:6466-6470; Lebkowski et al. (1988) Mol Cell Biol 8:3988-3996).

Vectors that contain both a promoter and a cloning site into which a polynucleotide can be operatively linked are well-known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potentially inappropriate alternative translation initiation codons or other sequences that may interfere with or reduce expression, either at the level of transcription or translation. Alternatively, consensus ribosome binding sites can be inserted immediately 5′ of the start codon to enhance expression. Examples of suitable vectors are viruses, such as baculovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

Among these are several non-viral vectors, including DNA/liposome complexes, and targeted viral protein DNA complexes. To enhance delivery to a cell, the nucleic acid or proteins of this invention can be conjugated to antibodies or binding fragments thereof which bind cell surface antigens, e.g, TCR, CD3 or CD4. Liposomes that also comprise a targeting antibody or fragment thereof can be used in the methods of this invention. The present invention also provides the targeting complexes for use in the methods disclosed herein.

Polynucleotides can be inserted into vector genomes using methods well-known in the art. For example, insert and vector DNA can be contacted, under suitable conditions, with a restriction enzyme to create complementary ends on each molecule that can pair with each other and be joined together with a ligase. Alternatively, synthetic nucleic acid linkers can be ligated to the termini of restricted polynucleotide. These synthetic linkers contain nucleic acid sequences that correspond to a particular restriction site in the vector DNA. Additionally, an oligonucleotide containing a termination codon and an appropriate restriction site can be ligated for insertion into a vector containing, for example, some or all of the following: a selectable marker gene, such as the neomycin gene for selection of stable or transient transfectants in mammalian cells; enhancer/promoter sequences from the immediate early gene of human CMV for high levels of transcription: transcription termination and RNA processing signals from SV40 for mRNA stability; SV40 polyoma origins of replication and ColEI for proper episomal replication; versatile multiple cloning sites; and T7 and SP6 RNA promoters for in vitro transcription of sense and antisense RNA. Other means are well-known and available in the art.

As used herein, the term “expression” refers to the process by which polynucleotides are transcribed into mRNA and translated into peptides, polypeptides, or proteins. If the polynucleotide is derived from genomic DNA expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook et al. (1989), supra). Similarly, a eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well-known in the art, for example, the methods described above for constructing vectors in general.

The terms “major histocompatibility complex” or “MHC” refers to a complex of genes encoding cell-surface molecules that are required for antigen presentation to immune effector cells, such as T cells and for rapid graft rejection. In humans, the MHC complex is also known as the HLA complex. The proteins encoded by the MHC complex are known as “MHC molecules” and are classified into “class I” and “class II” MHC molecules. Class I MHC molecules include membrane heterodimeric proteins made up of an a chain encoded in the MHC associated noncovalently with β2-microglobulin. Class I WIC molecules are expressed by nearly all nucleated cells and have been shown to function in antigen presentation to CD8+ T cells. Class I molecules include HLA-A, -B, and -C in humans. Class II MHC molecules also include membrane heterodimeric proteins consisting of noncovalently associated and J3 chains. Class II MHCs are known to function in CD4+ T cells and, in humans, include HLA-DP, -DQ, and DR. The term “MHC restriction” refers to a characteristic of T cells that permits them to recognize antigen only after it is processed and the resulting antigenic peptides are displayed in association with either a class I or class II MHC molecule. Methods of identifying and comparing MHC are well-known in the art and are described in Allen M. et al. (1994) Human Imm. 40:25-32: Santamaria P. et al. (1993) Human Imm. 37:39-50: and Hurley C. K. et al. (1997) Tissue Antigens 50:401-415.

The term “sequence motif” refers to a pattern present in a group of molecules (e.g., amino acids or nucleotides). For instance, in one embodiment, the present invention provides for identification of a sequence motif among peptides present in an antigen. In some embodiments, a typical pattern may be identified by characteristic amino acid residues, such as hydrophobic, hydrophilic, basic, acidic, and the like.

The term “peptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc.

The term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

The term “solid phase support” is used as an example of a “carrier” and is not limited to a specific type of support. Rather a large number of supports are available and are known to one of ordinary skill in the art. Solid phase supports include silica gels, resins, derivatized plastic films, glass beads, cotton, plastic beads, and alumina gels. A suitable solid phase support may be selected on the basis of desired end use and suitability for various synthetic protocols. For example, for peptide synthesis, a solid phase support can refer to resins, such as polystyrene (e.g., PAM-resin obtained from Bachem Inc., Peninsula Laboratories, etc.), POLYHIPE® resin (obtained from Aminotech, Canada), polyamide resin (obtained from Peninsula Laboratories), polystyrene resin grafted with polyethylene glycol (TentaGel®, Rapp Polymere, Tubingen, Germany), polydimethylacrylamide resin (obtained from Milligenl Biosearch, California), and the like. In a preferred embodiment for peptide synthesis, solid phase support refers to polydimethylacrylamide resin.

The term “aberrantly expressed” refers to polynucleotide sequences in a cell or tissue which are differentially expressed (either over-expressed or under-expressed) when compared to a different cell or tissue whether or not of the same tissue type, e.g., lung tissue versus lung cancer tissue.

The term “host cell” or “recipient cell” is intended to include any individual cell or cell culture which can be or have been recipients for vectors or the incorporation of exogenous nucleic acid molecules, polynucleotides and/or proteins. It also is intended to include progeny of a single cell, and the progeny may not necessarily be completely identical (in morphology or in genomic or total DNA complement) to the original parent cell due to natural, accidental or deliberate mutation. The cells may be prokaryotic or eukaryotic, and include but are not limited to bacterial cells, yeast cells, animal cells, and mammalian cells, e.g., murine, rat, simian or human.

An “antibody” is an immunoglobulin molecule capable of binding an antigen. As used herein, the term encompasses not only intact immunoglobulin molecules, but also anti-idiotypic antibodies, mutants, fragments, fusion proteins, humanized proteins and modifications of the immunoglobulin molecule that comprise an antigen recognition site of the required specificity.

An “antibody complex” is the combination of antibody and its binding partner or ligand.

A “native antigen” is a polypeptide, protein or a fragment containing an epitope, which induces an immune response in the subject.

The term “isolated” means separated from constituents, cellular and otherwise, in which the polynucleotide, peptide, polypeptide, protein, antibody, or fragments thereof: are normally associated with in nature. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the present inventions disclosed herein, it is to be understood that all of the above embodiments for each of the compositions disclosed below and under the appropriate conditions, are provided according to the present invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

The term “composition” is intended to mean a combination of active agent and another compound or composition, inert (for example, a detectable agent carrier, solid support or label) or active, such as an adjuvant.

The term “pharmaceutical composition” is intended to include the combination of an active agent with a carrier, inert or active, making the composition suitable for diagnostic or therapeutic use in vitro, in vivo, or ex vivo.

The term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI, 15th Ed. (Mack Publ. Co., Easton (1975)).

The term “inducing an immune response in a subject” is a term well understood in the art and intends that an increase of at least about 2-fold, more preferably at least about 5-fold, more preferably at least about 10-fold, more preferably at least about 100-fold, even more preferably at least about 500-fold, even more preferably at least about 1000-fold or more in an immune response to an antigen (or epitope) can be detected (measured), after introducing the antigen (or epitope) into the subject, relative to the immune response (if any) before introduction of the antigen (or epitope) into the subject. An immune response to an antigen (or epitope), includes, but is not limited to, production of an antigen-specific (or epitope-specific) antibody, and production of an immune cell expressing on its surface a molecule which specifically binds to an antigen (or epitope). Methods of determining whether an immune response to a given antigen (or epitope) has been induced are well-known in the art. For example, antigen-specific antibody can be detected using any of a variety of immunoassays known in the art, including, but not limited to, ELISA, wherein, for example, binding of an antibody in a sample to an immobilized antigen (or epitope) is detected with a detectably-labeled second antibody (e.g., enzyme-labeled mouse anti-human Ig antibody). Immune effector cells specific for the antigen can be detected using any of a variety of assays known to those skilled in the art, including, but not limited to, FACS, or, in the case of CTLs, ⁵¹CR-release assays, or ³H-thymidine uptake assays.

A “neoepitope” or “neoantigen” is intended to mean a unique, new epitope to a specific cancer, tumor, or cell thereof, which arises as a consequence of the accumulation of random mutations from aberrant DNA replication and/or repair; a hallmark of many cancers. Accordingly, neoepitopes/neoantigens can also be patient/subject specific.

The term “substantially free of endotoxin” means that there is less endotoxin per dose of cell fusions than is allowed by the FDA for a biologic, which is a total endotoxin of 5 EU/kg body weight per day.

The term “substantially free for mycoplasma and microbial contamination” means negative readings for the generally accepted tests know to those skilled in the art. For example, mycoplasm contamination is determined by subculturing a cell sample in broth medium and distributed over agar plates on day 1, 3, 7, and 14 at 37° C. with appropriate positive and negative controls. The product sample appearance is compared microscopically, at 100×, to that of the positive and negative control. Additionally, inoculation of an indicator cell culture is incubated for 3 and 5 days and examined at 600× for the presence of mycoplasmas by epifluorescence microscopy using a DNA-binding fluorochrome. The product is considered satisfactory if the agar and/or the broth media procedure and the indicator cell culture procedure show no evidence of mycoplasma contamination.

The sterility test to establish that the product is free of microbial contamination can be based on the U.S. Pharmacopedia Direct Transfer Method. This procedure requires that a pre-harvest medium effluent and a pre-concentrated sample be inoculated into a tube containing tryptic soy broth media and fluid thioglycollate media. These tubes are observed periodically for a cloudy appearance (turpidity) for a 14-day incubation. A cloudy appearance on any day in either medium indicate contamination, with a clear appearance (no growth) testing substantially free of contamination.

As used herein, a “sample” or “nucleic acid sample” can refer to any substance containing or presumed to contain nucleic acid. The sample can be a biological sample obtained from a subject. The nucleic acids can be RNA, DNA, e.g., genomic DNA, mitochondrial DNA, viral DNA, synthetic DNA, or cDNA reverse transcribed from RNA The nucleic acids in a nucleic acid sample generally serve as templates for extension of a hybridized primer. In some embodiments, the biological sample is a liquid sample. The liquid sample can be whole blood, plasma, serum, ascites, cerebrospinal fluid, sweat, urine, tears, saliva, buccal sample, cavity rinse, or organ rinse. The liquid sample can be an essentially cell-free liquid sample (e.g., plasma, serum, sweat, urine, tears, etc.). In other embodiments, the biological sample can be a solid biological sample, e.g., feces or tissue biopsy, e.g., a tumor biopsy. A sample can also comprise in vitro cell culture constituents (including, but not limited to, conditioned medium resulting from the growth of cells in cell culture medium, recombinant cells and cell components).

“Nucleotides” can be biological molecules that can form nucleic acids. Nucleotides can have moieties that contain not only the known purine and pyrimidine bases, but also other heterocyclic bases that have been modified. Such modifications include methylated purines or pyrimidines, acylated purines or pyrimidines, alkylated riboses, or other heterocycles. In addition, the term “nucleotide” includes those moieties that contain hapten, biotin, or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the like.

“Nucleotides” can also include locked nucleic acids (LNA) or bridged nucleic acids (BNA). BNA and LNA generally refer to modified ribonucleotides wherein the ribose moiety is modified with a bridge connecting the 2′ oxygen and 4′ carbon. Generally, the bridge “locks” the ribose in the 3′-endo (North) conformation, which is often found in the A-form duplexes. The term “locked nucleic acid” (LNA) generally refers to a class of BNAs, where the ribose ring is “locked” with a methylene bridge connecting the 2′-0 atom with the 4′-C atom LNA nucleosides containing the six common nucleobases (T, C, G, A, U and mC) that appear in DNA and RNA are able to form base-pairs with their complementary nucleosides according to the standard Watson-Crick base pairing rules. Accordingly, BNA and LNA nucleotides can be mixed with DNA or RNA bases in an oligonucleotide whenever desired. The locked ribose conformation enhances base stacking and backbone pre-organization. Base stacking and backbone pre-organization can give rise to an increased thermal stability (e.g., increased Tm) and discriminative power of duplexes. LNA can discriminate single base mismatches under conditions not possible with other nucleic acids. Locked nucleic acid is disclosed for example in WO 99/14226. Nucleotides can also include modified nucleotides, such as those described in European Patent Application No. EP1995330.

The terms “polynucleotides”, “nucleic acid”, “nucleotides” and “oligonucleotides” can be used interchangeably. They can refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “target polynucleotide” generally refers to a polynucleotide of interest under study. In certain embodiments, a target polynucleotide contains one or more sequences that are of interest and under study. A target polynucleotide can comprise, for example, a genomic sequence. The target polynucleotide can comprise a target sequence whose presence, amount, and/or nucleotide sequence, or changes in these, are desired to be determined.

The term “genomic sequence” generally refers to a sequence that occurs in a genome. Because RNAs are transcribed from a genome, this term encompasses sequence that exist in the nuclear genome of an organism, as well as sequences that are present in a cDNA copy of an RNA (e.g., an mRNA) transcribed from such a genome.

The terms “anneal”, “hybridize” or “bind,” can refer to two polynucleotide sequences, segments or strands, and can be used interchangeably and have the usual meaning in the art. Two complementary sequences (e.g., DNA and/or RNA) can anneal or hybridize by forming hydrogen bonds with complementary bases to produce a doublestranded polynucleotide or a double-stranded region of a polynucleotide.

The term “complementary” generally refers to a relationship between two antiparallel nucleic acid sequences in which the sequences are related by the base-pairing rules: A pairs with Tor U and C pairs with G. A first sequence or segment that is “perfectly complementary” to a second sequence or segment is complementary across its entire length and has no mismatches. A first sequence or segment is “substantially complementary” to a second sequence of segment when a polynucleotide consisting of the first sequence is sufficiently complementary to specifically hybridize to a polynucleotide consisting of the second sequence.

The term “duplex” or “duplexed” can describe two complementary polynucleotides that are base-paired, i.e., hybridized together.

As used herein, “amplification” of a nucleic acid sequence generally refers to in vitro techniques for enzymatically increasing the number of copies of a target sequence.

Amplification methods include both asymmetric methods (in which the predominant product is single-stranded) and conventional methods (in which the predominant product is double-stranded). A “round” or “cycle” of amplification generally refers to a PCR cycle in which a double stranded template DNA molecule is denatured into single-stranded templates, forward and reverse primers are hybridized to the single stranded templates to form primer/template duplexes, primers are extended by a DNA polymerase from the primer/template duplexes to form extension products. In subsequent rounds of amplification the extension products are denatured into single stranded templates and the cycle is repeated.

The terms “template”, “template strand”, “template DNA” and “template nucleic acid” can be used interchangeably herein to refer to a strand of DNA that is copied by an amplification cycle.

The term “denaturing,” as used herein, generally refers to the separation of a nucleic acid duplex into two single strands.

The term “extending”, as used herein, generally refers to the extension of a primer hybridized to a template nucleic acid by the addition of nucleotides using an enzyme, e.g., a polymerase.

A “primer” is generally a nucleotide sequence (e.g., an oligonucleotide), generally with a free 3′-OH group, that hybridizes with a template sequence (such as a target polynucleotide, or a primer extension product) and is capable of promoting polymerization of a polynucleotide complementary to the template. A primer can be, for example, a sequence of the template (such as a primer extension product or a fragment of the template created following RNase cleavage of a template-DNA complex) that is hybridized to a sequence in the template itself (for example, as a hairpin loop), and that is capable of promoting nucleotide polymerization. Thus, a primer can be an exogenous (e.g., added) primer or an endogenous (e.g., template fragment) primer.

The terms “determining”, “measuring”, “evaluating”, “assessing,” “assaying,” and “analyzing” can be used interchangeably herein to refer to any form of measurement and include determining of an element is present or not. These terms can include both quantitative and/or qualitative determinations. Assessing may be relative or absolute.

The term “assessing the presence of” can include determining the amount of something present, as well as determining whether it is present or absent.

The term “Tm” generally refers to the melting temperature of an oligonucleotide duplex at which half of the duplexes remain hybridized and half of the duplexes dissociate into single strands. See Sambrook and Russell (200 1, Molecular Cloning: A Laboratory Manual, 3.sup.rd ed., Cold Spring Harbor Press, Cold Spring Harbor N.Y., ch. 10).

The term “free in solution” can describe a molecule, such as a polynucleotide, that is not bound or tethered to a solid support.

The term “genomic fragment” can refer to a region of a genome, e.g., an animal or plant genome such as the genome of a human, monkey, rat, fish or insect or plant. A genomic fragment may or may not be adaptor ligated. A genomic fragment may be adaptor ligated (in which case it has an adaptor ligated to one or both ends of the fragment, to at least the 5′ end of a molecule), or non-adaptor ligated.

The term “ligating” can refer to the enzymatically catalyzed joining of the terminal nucleotide at the 5′ end of a first DNA molecule to the terminal nucleotide at the 3′ end of a second DNA molecule.

A “primer binding site” can refer to a site to which a primer hybridizes in an oligonucleotide or a complementary strand thereof.

The term “separating”, as used herein, can refer to physical separation of two elements (e.g., by size, affinity, degradation of one element etc.).

The term “sequencing”, as used herein, can refer to a method by which the identity of at least 10 consecutive nucleotides (e.g., the identity of at least 20, at least 50, at least 100, at least 200, or at least 500 or more consecutive nucleotides) of a polynucleotide are obtained.

The term “adaptor-ligated”, as used herein, can refer to a nucleic acid that has been ligated to an adaptor. The adaptor can be ligated to a 5′ end or a 3′ end of a nucleic acid molecule, or can be added to an internal region of a nucleic acid molecule.

The term “bridge PCR” can refer to a solid-phase polymerase chain reaction in which the primers that are extended in the reaction are tethered to a substrate by their 5′ ends. During amplification, the amplicons form a bridge between the tethered primers. Bridge PCR (which may also be referred to as “cluster PCR”) is used in Illumina's Solexa platform. Bridge PCR and Illumina's Solexa platform are generally described in a variety of publications, e.g., Gudmundsson et al (Nat. Genet. 2009 41: 1122-6), Out et al (Hum. Mutat. 2009 30:1703-12) and Turner (Nat. Methods 2009 6:315-6), U.S. Pat. No. 7,115,400, and published application numbers US20080160580 and US20080286795.

### OTHER EMBODIMENTS

While the present invention has been described in conjunction with the detailed description thereof, the foregoing description is intended to illustrate and not limit the scope of the present invention, which is defined by the scope of the appended claims. Other aspects, advantages, and modifications are within the scope of the following claims.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web and/or the National Center for Biotechnology Information (NCBI) on the world wide web.

### EQUIVALENTS

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below.

Those skilled in the art will recognize, or be able to ascertain using no more than routine experimentation, many equivalents to the specific embodiments of the present invention described herein. Such equivalents are intended to be encompassed by the following claims. In addition, the materials, methods, and examples described herein are illustrative only and are not intended to be limiting. Other features and advantages of the present invention will be apparent from encompassed by the following detailed description and claims.

What is claimed is:

1. A cell population comprising a dendritic cell fused to vehicle comprising a cDNA expression library derived from one or more tumor cells, optionally wherein the tumor cell and dendritic cell are autologous.

2. The cell population of claim 1, wherein the tumor cell is cultured in vitro.

3. The cell population of claim 2, wherein the cells are cultured using a 3D cell culture.

4. The cell population of any one of claims 1-3, wherein the tumor cells are a spheroid or organoid.

5. The cell population of any one of claims 1-4, wherein the vehicle is a MEW I/II null cell, optionally wherein the MHC I/II null cell is a fibroblast or a cancer cell line.

6. The cell population of any one of claims 1-5, wherein the vehicle is 1) a vesicle or 2) a polymeric nanoparticle (NP), optionally wherein the NP is a dual NP comprising a DNA expression vector-cationic peptide nanocomplex (NC) surrounded by a polymeric NP.

7. The cell population of claim 6, wherein the polymer NP is a diblock polymeric NP or a tetrablock polymeric NP.

8. The cell population of any one of claims 1-7, wherein the cDNA expression library is one or more defined tumor antigens and/or neoantigens, optionally wherein the tumor antigens and/or neoantigens are identified using RNA-seq.

9. A method of treating a tumor in a patient comprising administering to the patient a composition comprising a cell population comprising a dendritic cell obtained from the patient fused to a vehicle comprising a cDNA expression library derived from a tumor cell, optionally wherein the tumor cell and dendritic cell are autologous.

10. The method of claim 9, wherein the tumor cell is cultured in vitro, optionally wherein the culture is a 3D cell culture.

11. The method of claim 9 or 10, wherein the tumor cells are a spheroid or organoid.

12. The method of any one of claims 9-11, wherein the vehicle is 1) a vesicle or 2) a polymeric nanoparticle (NP), optionally wherein the NP is a dual NP comprising a DNA expression vector-cationic peptide nanocomplex (NC) surrounded by a polymeric NP.

13. The method of claim 12, wherein the polymer NP is a diblock polymeric NP or a tetrablock polymeric NP.

14. The method of any one of claims 9-13, wherein the cDNA expression library is one or more defined tumor antigens and/or neoantigens, optionally wherein the tumor antigens and/or neoantigens are identified using RNA-seq.

15. The method of any one of claims 9-14, wherein the tumor is a solid tumor or a hematologic malignancy, optionally wherein the solid tumor is a breast tumor or a renal tumor or wherein the hematologic malignancy is acute myeloid leukemia (AML) or multiple myeloma (MM).

16. The method of any one of claims 9-15, further comprising administering to the patient an indoleamine-2,3-dioxygenase (IDO) inhibitor and/or a hypomethylating agent.

17. The method of any one of claims 9-16, further comprising administering to the patient an immunomodulatory agent.

18. The method of claim 17, wherein the immunomodulatory agent is lenalidomide, pomalinomide, or apremilast.

19. The method of any one of claims 9-18, further comprising administering to the patient a checkpoint inhibitor.

20. The method of claim 19, wherein the checkpoint inhibitor is a PD1, PDL1, PDL2, TIM3, or LAG3 inhibitor.

21. The method of claim 19, wherein the checkpoint inhibitor is a PD1, PDL1, TIM3, or LAG3 antibody.

22. The method of any one of claims 9-21, further comprising administering to the patient an agent that target regulatory T cells.

23. The method of any one of claims 9-22, further comprising administering to the patient a TLR agonist, CPG ODN, polyIC, or tetanus toxoid.

24. The method of claim 16, wherein the hypermethylating agent is GO-203 or decitabine.

25. The method of claim 16, wherein the IDO inhibitor is INB024360 or 1-MDT.

26. A method of producing a fused cell population, comprising: providing a population a population of dendritic cells (DC) or hyperactive dendritic cells and a vehicle comprising a cDNA expression library derived from a tumor cell and mixing the population of dendritic cells and the vehicle under conditions capable of mediating fusion of the dendritic cells and vehicle to produce a fused cell population, optionally wherein the tumor cell and dendritic cell are autologous.

27. The method of claim 26, wherein the population of hyperactive dendritic cells is produced by:
a. contacting a population of dendritic cells with a composition comprising CpG DNA or LPS for a first period of time to produce a primed population of dendritic cells; and
b. contacting the primed population of dendritic cells with a composition comprising oxidized phospholipids for a second period of time to produce a population of hyperactive dendritic cells.

28. The method of claim 26 or 27, wherein the tumor cell is cultured in vitro.

29. The method of any one of claims 26-28, wherein the cells are cultured using a 3D cell culture.

30. The method of any one of claims 26-29, wherein the tumor cells are a spheroid or organoid.

31. The method of any one of claims 26-30, wherein the vehicle is 1) a vesicle or 2) a polymeric nanoparticle (NP), optionally wherein the NP is a dual NP comprising a DNA expression vector-cationic peptide nanocomplex (NC) surrounded by a polymeric NP.

32. The method of any one of claims 26-31, wherein the polymer NP is a diblock polymeric NP or a tetrablock polymeric NP.

33. The method of any one of claims 26-32, wherein the cDNA expression library is one or more defined tumor antigens and/or neoantigens, optionally wherein the tumor antigens and/or neoantigens are identified using RNA-seq.

34. The method of any one of claims 26-33, wherein the dendritic cells and the vehicles are at a ratio of 10:1 to 3:1.

35. The method of any one of claims 26-34 wherein the conditions capable of mediating fusion include a fusion agent, optionally wherein the fusion agent is polyethylene glycol (PEG).

36. The method of any one of claims 26-35, further comprising contacting the fused cell population with an indoleamine-2,3-dioxygenase (IDO) inhibitor.

37. A cell population produced by the method of any one of claims 26-36.

38. The cell population of claim 37, wherein the cell population is substantially free of endotoxin, microbial contamination and mycoplasma, optionally wherein the viability of the cell population is at least 80%.

39. A cell population comprising a dendritic cell fused to tumor cell, wherein the tumor cell is derived from 3D culturing a tumor cell obtained from a patient, optionally wherein the 3D culturing produces a tumor spheroid or organoid.

40. The cell population of claim 39, wherein the dendritic cell and the tumor cell are autologous.

41. A vaccine composition comprising the cell population of any one of claims 37-40.

42. A method of treating a tumor in a patient comprising administering to the patient a composition comprising a cell population comprising a dendritic cell fused to tumor cell, wherein the tumor cell is derived from 3D culturing a tumor cell obtained from the patient.

43. The method of claim 42, wherein the tumor is a solid tumor or a hematologic malignancy.

44. The method of claim 43, wherein said solid tumor is a breast tumor, or a renal tumor.

45. The method of claim 43, wherein the hematologic malignancy is acute myeloid leukemia (AML) or multiple myeloma (MM).

46. The method of any one of claims 42-45, further comprising administering to the patient an indoleamine-2,3-dioxygenase (IDO) inhibitor and/or a hypomethylating agent.

47. The method of any one of claims 42-46, further comprising administering to the patient an immunomodulatory agent.

48. The method of claim 47, wherein the immunomodulatory agent is lenalidomide pomalinomide, or apremilast.

49. The method of any one of claims 42-48, further comprising administering to the patient a checkpoint inhibitor.

50. The method of claim 49, wherein the checkpoint inhibitor is a PD1, PDL1, PDL2, TIM3, or LAG3 inhibitor.

51. The method of claim 49, wherein the checkpoint inhibitor is a PD1, PDL1, TIM3, or LAG3 antibody.

52. The method of any one of claims 42-51, further comprising administering to the patient an agent that target regulatory T cells.

53. The method of any one of claims 42-51, further comprising administering to the patient a TLR agonist, CPG ODN, polyIC, or tetanus toxoid.

54. The method of claim 46, wherein in the hypermethylating agent is GO-203 or decitabine.

55. The method of claim 46, wherein the IDO inhibitor is INB024360 or 1-MDT.

56. A method of producing a fused cell population, comprising: providing a population a population of dendritic cells (DC) or hyperactive dendritic cells and a population of tumor spheroids or organoids and mixing the population of dendritic cells and the population spheroids or organoids under conditions capable of mediating fusion of the dendritic cells and spheroids or organoids to produce a fused cell population, optionally wherein the tumor spheroids or organoids and dendritic cells are autologous.

57. The method of claim 56, wherein the population of hyperactive dendritic cells is produced by:
a. contacting a population of dendritic cells with a composition comprising CpG DNA or LPS for a first period of time to produce a primed population of dendritic cells; and
b. contacting the primed population of dendritic cells with a composition comprising oxidized phospholipids for a second period of time to produce a population of hyperactive dendritic cells.

58. The method of claim 56, wherein the spheroids or organoids are derived from a patient's tumor.

59. The method of any one of claims 56-58, wherein the dendritic cells and the spheroids or organoids are at a ratio of 10:1 to 3:1.

60. The method of any one of claims 56-59, wherein the conditions capable of mediating fusion include a fusion agent.

61. The method of claim 60, wherein the fusion agent is polyethylene glycol (PEG).

62. The method of any one of claims 56-61, further comprising contacting the fused cell population with an indoleamine-2,3-dioxygenase (DO) inhibitor.

63. The cell population produced by the method of any one of claims 56-62.

64. The cell population of claim 63, wherein the cell population is substantially free of endotoxin, microbial contamination and mycoplasma.

65. The cell population of claim 63 or 64, wherein the viability of the cell population is at least 80%.

66. A vaccine composition comprising the cell population of any one of claims 56-65.
